(19) United States
(12) Patent Application Publication
(10) Pub. No.: US 2009/0104249 A1
(43) Pub. Date: Apr. 23, 2009

(54) Exotoxin Inhibitory Factor

(75) Inventors: Patrick M. Schlievert, Edina, MN (US); Marnie L. Peterson, Golden Valley, MN (US)

Correspondence Address: FISH & RICHARDSON P.C., P.O. BOX 1022, MINNEAPOLIS, MN, 55440-1022, US

(73) Assignee: REGENTS OF THE UNIVERSITY OF MINNESOTA, Saint Paul, MN (US)

(21) Appl. No.: 11/995,411

(22) PCT Filed: Dec. 23, 2005
(86) PCT No.: PCT/US05/46948

§ 371 Date: Nov. 13, 2008

Related U.S. Application Data
(60) Provisional application No. 60/638,958, filed on Dec. 23, 2004.

(51) Int. Cl. A61K 9/00; C07K 14/00; A61K 9/70; A61K 38/16; A61P 31/04

(52) U.S. Cl. 424/431; 530/350; 424/422; 424/447; 424/484; 514/12

(57) ABSTRACT

The invention provides an exotoxin inhibitory factor, compositions containing the factor, methods of using the compositions.

### TECHNICAL FIELD

This invention relates to bacterial toxicity, and more particularly to inhibition of bacterial exotoxin production.

### BACKGROUND

Bacterial toxicity, e.g., toxic shock syndrome, remains a social and economic problem throughout the world. Indeed, in some geographic areas the incidence of such bacterial toxicity (e.g., menstrual Staphylococcal aureus-associated toxic shock) is increasing. Thus there is an urgent need to control the growth of relevant bacteria and/or the production by such bacteria of the etiological agents (e.g., exotoxins) of the toxicity.

### SUMMARY

The invention is based on the finding that a factor in blood and human menses inhibited the production of toxin shock syndrome toxin 1 (TSST-1) and α-hemolysin by S. aureus bacteria.

The invention provides an isolated factor that is identical to a factor in blood that inhibits the production of an exotoxin by a bacterium. The factor in blood can include a protein with an isoelectric point (pI) of 7-8. The bacterium can be a Staphylococcus, e.g., S. aureus and the exotoxin can be, for example, toxic shock syndrome toxin 1 (TSST-1), α-hemolysin, or a staphylococcal enterotoxin.

The invention also provides a composition that includes: a medical or hygienic device; and an isolated factor that is identical to a factor in blood that inhibits the production of an exotoxin by a bacterium. All or part of the surface of the device can be coated with or impregnated with the isolated factor. The device can be, e.g., a vaginal tampon, a surgical suture, a surgical bandage, a surgical dressing, an osmotic pump, an ostomy device, or a matrix for tissue repair.

Also featured by the invention is a composition that includes: a pharmaceutical carrier or excipient; and an isolated factor that is identical to a factor in blood that inhibits the production of an exotoxin by a bacterium.

Another aspect of the invention is a method of treating or preventing the symptoms of a bacterial infection. The method includes delivery to a tissue or organ of a vertebrate subject of an isolated factor that is identical to a factor in blood that inhibits the production of a bacterial exotoxin, wherein the tissue or organ is, or is in danger of being, infected with a bacterium.

The invention also embodies method of preventing the symptoms of a bacterial infection. The method includes: providing a medical or hygienic device; providing an isolated factor that is identical to a factor in blood that inhibits the production of a bacterial exotoxin; applying the device to a vertebrate subject; and administering to the vertebrate subject the isolated factor. The device can be coated or impregnated with the isolated factor and the applying of the device and the administration of the factor can occur simultaneously. The device can be any of those recited herein, e.g., a vaginal tampon.

Another embodiment of the invention is an article of manufacture that includes packaging material and, within the packaging material, a composition containing an isolated factor that is identical to a factor in blood that inhibits the production of a bacterial exotoxin, the packaging material including instructions that indicate that the composition can be used for preventing or inhibiting the symptoms of a bacterial infection.

As used herein, “prophylaxis” can mean complete prevention of the symptoms of a disease, a delay in onset of the symptoms of a disease, or a lessening in the severity of subsequently developed disease symptoms. As used herein, “therapy” can mean a complete abolishment of the symptoms of a disease or a decrease in the severity of the symptoms of the disease.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents, and other references mentioned herein are incorporated by reference in their entirety. The materials, methods, and examples disclosed herein are illustrative only and not intended to be limiting.

Other features and advantages of the invention, e.g., treating toxic shock syndrome, will be apparent from the following description and from the claims.

### DESCRIPTION OF DRAWINGS

FIGS. 1A and 1B are a pair of line graphs showing the concentrations of S. aureus bacteria (“Cells/ml”) and TSST-1 (in μg/ml) at various time points in cultures of S. aureus bacteria in either whole human blood (FIG. 1A) or beef heart medium (FIG. 1B).

FIG. 2 is a profile depicting the absorbance at 280 nm of fractions obtained from a reverse phase high pressure liquid chromatography (RP-HPLC) separation of a 0%-25% ethanol precipitated fraction of human menses.

FIG. 3 is a diagram of an isoelectric focusing (IEF) gel indicating the position of colored bands (shaded areas) and non-colored regions of the gels. The pH of eluates prepared from gel segments corresponding to the colored bands and non-colored regions cut out of the gel are indicated.

### DETAILED DESCRIPTION

The invention features a factor, compositions containing the factor, articles of manufacture including such compositions, and methods using the compositions for inhibiting the production of bacterial exotoxins. The methods can be applied to the treatment and or prophylaxis of a variety of bacterial (e.g., S. aureus) infections, including infections associated with the use of a variety of medical and/or hygienic devices. These infections include, for example, menstrual as well as non-menstrual S. aureus infections.

Menstrual toxic shock syndrome (mTSS) occurs primarily in young women of menstrual age who use tampons. Although the illness occurs within one to two days of onset or termination of menses, the peak time for onset is day 3 to 4. It has always been assumed that tampons collect menstrual blood, which provides a source of nutrients for toxic shock syndrome (TSS) causing Staphylococcus aureus (TSS S. aureus) to grow on the tampons and make toxic shock syndrome toxin-1 (TSST-1), the principal cause of mTSS. TSST-1 then likely crosses into the subject's blood circulatory system by an as yet unknown mechanism. TSS symptoms arise in the subject if she lacks antibodies capable of neutralizing TSST-1.

The environmental factors that regulate production of S. aureus exotoxins have been investigated. Work from the inventors' laboratory has shown that exotoxin is made by S. aureus at body temperature, at neutral pH, in the presence of protein, in the absence of high levels of glucose, and in the presence of at least 2% oxygen balanced with 7% carbon dioxide [Schlievert et al. (1983) J. Infect. Dis. 147(2): 236-242]. It was suggested that the reason tampons are associated with TSS is that they introduce oxygen into a typically anaerobic vaginal environment, with consequent stimulation of TSST-1 production [Wagner et al. (1984) Am. J. Obstet. Gynecol. 148(2):147-150]. More recent evidence indicates that, while intra-vaginal tampons do contain oxygen, they do not cause the vagina to be oxygenated. The instant invention is not limited by any particular mechanism by which production of TSST-1 by intra-vaginal S. aureus is induced and/or facilitated.

Various aspects of the invention are described below.

### Exotoxin-Inhibitory Factor (EIF)

The inventors have discovered that a factor present in mammalian blood inhibited the production of a variety of staphylococcal enterotoxins. e.g., TSST-1, SEB, SEC, and α-hemolysin. As used herein, an “exotoxin inhibitory factor” (“EIF”) is a factor that either completely ablates or reduces the amount of exotoxin produced by a bacterium. The EIF of the invention is a factor identical to the EIF in mammalian blood discovered by the inventors. Such a factor can be a single molecular entity. Alternatively, it can be composed of multiple (e.g., two, three, four, five, six, seven, eight, nine, ten or more) molecular entities. Moreover the entity can be any biological molecule, e.g., a protein (including, e.g., a glycoprotein or a lipoprotein), a carbohydrate, a lipid, a nucleic acid, or a small molecule such as a vitamin or hormone (peptide or other). Moreover, an entity can be a part, segment or fragment of any such biological molecule. It could be, for example, a lipid or carbohydrate moiety of a lipoprotein or a glycoprotein, respectively. It could also be for a moiety such as, for example, the heme group that is bound to hemoglobin. If, for example, both hemoglobin and heme were found to be active as EIF, either could be the entity. The factor can be used in a relatively crude form (e.g., as a culture supernatant), a semi-purified form, or a highly purified form. It will preferably be isolated.

An “isolated” factor as used herein refers to a factor which either has no naturally-occurring counterpart or has been separated or purified from components which naturally accompany it, e.g., in tissues such as skin, fat, pancreas, liver, spleen, ovary, testis, muscle, joint tissue, neural tissue, gastrointestinal tissue or tumor tissue; body fluids such as blood, serum, plasma, menses, semen, saliva, or urine; or secretions such as mucus, vaginal secretions, or pulmonary secretions. Typically, the factor is considered “isolated” when it is at least 70%, by dry weight, free from the other naturally-occurring organic molecules with which it is naturally associated. Preferably, a preparation of a factor of the invention is at least 80%, more preferably at least 90%, and most preferably at least 99%, by dry weight, the factor of the invention. Thus, for example, a preparation of factor x is at least 80%, more preferably at least 90%, and most preferably at least 99%, by dry weight, factor x. Since a factor that is chemically synthesized is, by its nature, separated from the components that naturally accompany it, the synthetic factor is “isolated.”

An isolated factor of the invention can be obtained, for example: by extraction from a natural source (e.g., from tissues such as skin, fat, pancreas, liver, spleen, ovary, testis, muscle, joint tissue, neural tissue, gastrointestinal tissue or tumor tissue; body fluids such as blood, serum, plasma, menses, semen, saliva, or urine; or secretions such as mucus, vaginal secretions, or pulmonary secretions); by, in the case of a polypeptide, expression of a recombinant nucleic acid encoding the polypeptide; or by chemical synthesis. A factor that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

Bacteria of interest include, without limitation, Staphylococci (e.g., S. aureus, S. intermedius, S. epidermidis, and other coagulase negative Staphylococci), Neisseriae (e.g., N. gonorrheae and N. meningitidis), Streptococci (e.g., Group A Streptococcus (e.g., S. pyogenes), Group B Streptococcus (e.g., S. agalactiae), Group C Streptococcus, Group G Streptococcus, S. pneumoniae, and viridans Streptococci), Chlamydia trachomatis, Treponemae (e.g., T. pallidum, T. pertenue, and T. cerateum), Haemophilus bacteria (e.g., H. ducreyi, H. influenzae, and H. aegyptius), Bordetellae (e.g., B. pertussis, B. parapertussis, and B. bronchiseptica), Gardnerella vaginalis, Bacillus (e.g., B. anthracis and B. cereus), and Clostridium (e.g. C. perfringens, C. septicum, C. novyi, and C. tetani), Escherichia coli, Vibrio cholerae, Salmonella bacteria (e.g., S. enteriditis, S. typhimurium, and S. typhi), Shigella bacteria, Mycobacteria, Francisella bacteria, Yersinia bacteria (e.g. Y. pestis), Burkholderia bacteria, Pseudomonas bacteria, and Brucella bacteria.

Exotoxins inhibited by EIF include, without limitation, TSST-1, Staphylococcal alpha, beta, gamma, and delta hemolysins, Streptococcal pyrogenic exotoxins (SPEs), streptolysin O, streptolysin S, Staphylococcal enterotoxins (SEs; such as SEA, SEB, SEC, or SEE), A-B toxins, diptheria exotoxin, cholera exotoxin, pertussis exotoxin, shiga toxin, shiga-like toxin, anthrax (B. anthracis) toxin, botulinal exotoxin, tetanus exotoxin, tracheal cytotoxin, Helicobacter toxins, alpha toxin (lecithinase), kappa toxin (collagenase), mu toxin (hyaluronidase), leukocidin, elastase and other proteases, a Panton Valentine leukocidin component (S or F component), a porin, Listeria monocytogenes cytolysin, aerolysin, Bacillus anthracis protective antigen, and nucleases.

The EIF can be that of a vertebrate, for example: a mammal such as a human, non-human primate (e.g., monkey), mouse, rat, hamster, gerbil, guinea pig, cow, sheep, goat, horse, pig, rabbit, dog, or cat; or a bird such as a chicken, turkey, canary, eagle, or hawk.

With respect to polypeptide EIF, the invention includes full-length immature (unprocessed) polypeptides, full-length mature polypeptides, and functional fragments of either. “Polypeptide” and “protein” are used interchangeably and mean any peptide-linked chain of amino acids, regardless of length or post-translational modification. As used herein, a “functional fragment” of a EIF is a fragment of the full-length, wild-type, EIF polypeptide that is shorter than the full-length, wild-type, mature EIF polypeptide but has at least 20% (e.g., at least: 20%; 30%; 40%; 50%; 60%; 70%; 80%; 85%; 90%; 95%; 98%; 99%; 99.5%; 99.8%; 100%; or even more) of the exotoxin-inhibitory activity of the full-length, wild-type, mature EIF polypeptide.

An EIF polypeptide of the invention will preferably have a pI (isoelectric point) of 6.7-8.2, e.g., 7.0-8.0.

The invention also features EIF polypeptides, or functional fragments thereof, with not more than 25 (e.g., not more than; 25; 20; 15; 12; 10; nine; eight; seven; six; five; four; three; two; or one) conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. A polypeptide (including a functional fragment) with one or more conservative substitutions has at least 20% (as above) of the exotoxin-inhibitory activity of the corresponding parent, unmutated polypeptide.

The polypeptides of the invention can be purified from natural sources (e.g., tissues such as skin, fat, pancreas, liver, spleen, ovary, testis, muscle, joint tissue, neural tissue, gastrointestinal tissue or tumor tissue; body fluids such as blood, serum, plasma, menses, semen, saliva, or urine; or secretions such as mucus, vaginal secretions, or pulmonary secretions). Smaller peptides (less than 100 amino acids long) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals (see below). See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to inhibit bacterial exotoxin production in a manner qualitatively identical to that of the EIF polypeptide functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The invention also provides nucleic acid molecules encoding the above-described EIF polypeptides. The nucleic acid molecules of the invention can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to coding sequences, e.g., they can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a mammal. The nucleic acids can be those of a vertebrate, e.g.: a mammal such as a human, non-human primate (e.g., monkey), mouse, rat, hamster, gerbil, guinea pig, cow, sheep, goat, horse, pig, rabbit, dog, or cat; or a bird such as a chicken, turkey, canary, eagle, or hawk. Combinations or modifications of the nucleotides within these types of nucleic acids are also encompassed by the invention.

In addition, the isolated nucleic acid molecules of the invention encompass segments that are not found as such in the natural state. Thus, the invention encompasses recombinant nucleic acid molecules incorporated into a vector (for example, a plasmid or viral vector) or into the genome of a heterologous cell (or the genome of a homologous cell, at a position other than the natural chromosomal location).

Techniques associated with detection or regulation of genes (e.g., hybridization) are well known to skilled artisans. Such techniques can be used to diagnose and/or treat disorders associated with EIF polypeptide expression, e.g., toxic shock. Hybridization can also be used as a measure of homology between two nucleic acid sequences. An EIF polypeptide-encoding nucleic acid sequence, or a portion thereof, can be used as a hybridization probe according to standard hybridization techniques. The hybridization of an EIF polypeptide nucleic acid probe to DNA or RNA from a test source (e.g., a mammalian cell) is an indication of the presence of the EIF polypeptide-encoding DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

The invention also encompasses: (a) vectors (see below) that contain any of the foregoing EIF polypeptide coding sequences and/or their complements (that is, “antisense” sequences); (b) expression vectors that contain any of the foregoing EIF polypeptide coding sequences operably linked to any transcriptional/translational regulatory elements (examples of which are given below) necessary to direct expression of the coding sequences; (c) expression vectors encoding, in addition to a EIF polypeptide, a sequence unrelated to the EIF polypeptide, such as a reporter, a marker, or a signal peptide fused to the hair growth polypeptide; and (d) genetically engineered host cells (see below) that contain any of the foregoing expression vectors and thereby express the nucleic acid molecules of the invention. As used herein, “operably linked” means incorporated into a genetic construct so that expression control sequences effectively control expression of a coding sequence of interest.

Recombinant nucleic acid molecules can contain a sequence encoding an EIF polypeptide or the EIF polypeptide with a heterologous signal sequence. The full-length EIF polypeptide, or a fragment thereof, may be fused to such heterologous signal sequences or to additional polypeptides, as described below. Similarly, the nucleic acid molecules of the invention can encode the mature form of the EIF polypeptide or a form that includes an exogenous polypeptide that facilitates secretion.

The transcriptional/translational regulatory elements referred to above include, but are not limited to inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences, for example, a sequence that functions as a marker or reporter. Examples of marker and reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being a EIF polypeptide and the second portion being, for example, the reporter described above or an Ig constant region or part of an Ig constant region, e.g., the CH2 and CH3 domains of IgG2a heavy chain. Other hybrids could include an antigenic tag or His tag to facilitate purification.

The expression systems that may be used for purposes of the invention include, but are not limited to, microorganisms such as bacteria (for example, E. coli and B. sublilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecule of the invention; insect cell systems infected with recombinant virus expression vectors (for example, baculovirus) containing a nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing an EIF polypeptide-encoding nucleotide sequence; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

### Uses of EIF

EIF can be utilized in many different ways. For example, an EIF can be a component of an injectable composition which is injected into or applied to an infected area. Whether provided dry or in solution, the compositions of the invention can be prepared for storage by mixing them with any one or more of a variety of pharmaceutically acceptable carriers, excipients or stabilizers known in the art [Remington's Pharmaceutical Sciences, 16th Edition, Osol, A. Ed. 1980]. Acceptable carriers, excipients, or stabilizers are nontoxic to recipients at the dosages and concentrations employed, and include: buffers, such as phosphate, citrate, and other non-toxic organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol, or sorbitol; salt-forming counterions such as sodium; and/or nonionic surfactants such as Tween, Pluronics, or PEG. Alternatively, the EIF can be a component of a cream or solution to be applied topically to an infected area or to an area at risk of being infected, optionally in combination with any known non-toxic delivery agent and/or penetrant.

The compositions of the invention can be administered orally or by intravenous infusion, or injected subcutaneously, intramuscularly, intrathecally, intraperitoneally, intrarectally, intravaginally, intranasally, intragastrically, intratracheally, or intrapulmonarily. The dosage required depends on the choice of the route of administration; the nature of the formulation; the nature of the patient's condition; the subject's size, weight, surface area, age, and sex; other drugs being administered; and the judgment of the attending physician. Suitable dosages are in the range of 0.01-100.0 mg/kg. Wide variations in the needed dosage are to be expected in view of the differing efficiencies of various routes of administration. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding an EIF polypeptide or functional fragment thereof can be delivered to cells in a mammalian subject. Expression of the coding sequence can be directed to any cell in the body of the subject but will preferably be directed to cells in, or in the vicinity of an infection. Uptake of nucleic acids by cells can be achieved by, for example, the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73, 479]. Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the EIF polypeptide or functional fragment of interest with an initiator methionine and optionally a targeting sequence is operably linked to a promoter or enhancer-promoter combination.

Short amino acid sequences can act as signals to direct proteins to specific intracellular compartments. Such signal sequences are described in detail in U.S. Pat. No. 5,827,516, incorporated herein by reference in its entirety.

Enhancers provide expression specificity in terms of time, location, and level. Unlike a promoter, an enhancer can function when located at variable distances from the transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. The coding sequence of the expression vector is operably linked to a transcription terminating region.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased bacterial exotoxin production) in a treated animal. As is well known in the medical arts, the dosage for any one subject depends upon many factors, including the subject's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

An ex vivo strategy can involve transfecting or transducing cells obtained from the subject with a polynucleotide encoding an EIF polypeptide or functional fragment-encoding nucleic acid sequences. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such transfected or transduced cells act as a source of the EIF polypeptide or functional fragment for as long as they survive in the subject.

The ex vivo methods include the steps of harvesting cells from a subject, culturing the cells, transducing them with an expression vector, and maintaining the cells under conditions suitable for expression of the EIF polypeptide or functional fragment. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells can then be lethally irradiated (if desired) and injected or implanted into the patient.

The EIF of the invention can also be used, optionally with other factors, as a culture medium supplement for in vitro where it is desired, for example, to grow cells (e.g., mammalian cells) with exotoxin-producing bacteria but to minimize toxic effects of the exotoxin on the cells. The EIF can also be used as a “positive control” in in vitro assays, e.g., for testing for other exotoxin inhibitory factors.

Other compositions of the invention include those containing an EIF and a medical or hygienic device. As used herein, a “medical or hygienic device” is a device that is inserted into a bodily canal of a vertebrate subject, inserted into a bodily cavity of a vertebrate subject, or applied to a tissue or organ of a vertebrate animal for the purpose of: (a) wound protection; (b) preventing or reducing unwanted, or overcoming restricted, release from the body of the vertebrate subject of a bodily fluid, bodily secretion, or excreta (e.g., blood, menses, urine, lymphatic fluid, cerebrospinal fluid, semen, saliva, vaginal secretions, mucus, or feces); (c) delivering a drug or some other therapeutic or prophylactic agent to a subject; (d) replacing absent or supplementing defective organ functions; or (e) maintaining the patency of a bodily canal (e.g., a blood vessel). Devices of interest include, without limitation: rectal devices such as suppositories, enemas, and catheters; nasal, tracheal, or esophageal delivery devices; vaginal devices such as vaginal tampons and contraceptive devices (e.g., diaphragms or intrauterine devices (IUDs)); venous, arterial, intracranial and other needles, catheters and stents; renal dialysis accesses; surgical bandages, sutures, or dressings; ostomy devices; natural and synthetic implantable tissue matrices (see, for example, U.S. Pat. No. 5,885,829 which is incorporated herein by reference in its entirety); pace makers and pace maker wires and leads; synthetic and natural prostheses such as hip and knee prostheses and heart valves; osmotic pumps (e.g., mini osmotic pumps) that are implanted in body cavity (e.g., the peritoneal cavity) and provide slow delivery of a drug or some other therapeutic or prophylactic agent.

In these compositions, the EIF and the device be provided separately. Thus, they can be provided in the form of a kit or article of manufacture, optionally also containing packaging materials. The device and the EIF can optionally be in separate containers. In the kit or article of manufacture there can optionally be instructions (e.g., on the packing materials or in a package insert) on how to apply the device and administer the EIF. The EIF in such compositions can be formulated as described above. Generally, however, the EIF and device will provided in a combined form. Thus, the EIF can be coated onto part, or all, of the surface of the device and/or impregnated into part, or all, of the body of the device.

As used herein, “applying a device” to a vertebrate subject means inserting all or part of the device into a bodily canal (e.g., a vagina) or bodily cavity (e.g., a peritoneum or pleural cavity) of the subject or placing all or part of the device in a touching relationship with the surface or within the body of a tissue or organ of the subject.

The invention also provides methods of using the device/EIF compositions. The devices and EIF can be used separately or combined prior to use. If used separately, the EIF can be administered by any of the methods described above and the device applied to the vertebrate subject as described above. Where the device and EIF are provided in a combined form, the composition is applied in same manner as the device alone.

In any of the above described methods, the subject can be a vertebrate, for example: a mammal such as a human, non-human primate (e.g., monkey), mouse, rat, hamster, gerbil, guinea pig, cow, sheep, goat, horse, pig, rabbit, dog, or cat; or a bird such as a chicken, turkey, canary, eagle, or hawk.

The following examples are meant to illustrate, not limit, the invention.

### EXAMPLE 1

### The Effect of Rabbit Blood on the Growth of TSS S. aureus and TSST-1 Production by S. aureus in a Vaginal Tampon

The effect of blood on the growth of TSS S. aureus and TSST-1 production was studied in two different in vitro systems. In the first study the typical mTSS isolate MN8 was used; this organism represents the major class of vaginal TSS isolates.

In the first system, 5 ml of blood from a normal healthy rabbit was added to one side of a Tambrands™ tampon that had been inserted into dialysis tubing (12,000 to 14,000 molecular weight cut-off). The volume of blood added was sufficient to soak only one side of the tampon, leaving the opposite side free, visually at least, of blood. The tampon was then inoculated with cells of the typical mTSS S. aureus MN8 so as to distribute the bacteria as uniformly as possible throughout the tampon. The dialysis tubing was sealed and then immersed in liquid Todd Hewitt agar (1% agar) in a large glass test tube. The agar was allowed to solidify and the test tube was incubated for 18 hours at 37° C. The dialysis tube was removed from the glass tube, and the tampon removed from the diaysis tubing. Approximately equal sized segments were excised from both the side of the tampon to which blood had been added and the side that was free of blood (four segments per side). The levels of TSST-1 on the various segments of the tampon were determined by a quantitative immunoblotting procedure using a TSST-1-specific polyclonal antibody. While very little (about 10 ng per segment) TSST-1 was made in the tampon segments that contained blood, segments that were free of blood contained high levels (about 9.4 μg per segment). These data suggested that the blood contained an active substance that inhibited TSST-1 production.

### EXAMPLE 2

### The Effect of Human Menses on the Growth of TSS S. aureus and TSST-1 Production by TSS S. aureus in a Vaginal Tampon

A soiled tampon from a normal healthy woman lacking antibody specific for TSST-1 was evaluated for TSS S. aureus and TSST-1 levels. The tampon was cut into 24×0.2 gm external sections and 6×0.4 gm internal sections. Each section was suspended in 1.0 ml of water and the fluid was expressed from it. Measurements of the numbers of TSS S. aureus bacteria recovered, the A₄₁₀ of the expressed fluids or a dilution of the expressed fluids (as an indication of blood contamination), the pH of the expressed fluid, and the amounts of TSST-1 recovered were made. Approximately 10⁹ TSS S. aureus organisms were recovered from the whole tampon. Interestingly, the regions of the tampon that contained detectable levels of TSST-1 were those that contained minimal or no detectable menstrual blood. On the other hand, in regions that were significantly contaminated with blood, no TSST-1 was detectable. The numbers of S. aureus organisms in the regions in which substantial TSST-1 levels were detected were not significantly different from the numbers in the regions in which no TSST-1 was detectable. These findings, which are consistent with the rabbit blood study described above, suggest that human blood contains a substance that actively inhibits TSST-1 production. In tests of tampons from two additional persons, similar findings were obtained. In a test of one soiled tampon, α-hemolysin as well as TSST-1 production was tested for; inhibition of the production of both TSST-1 and α-hemolysin was seen in regions of the tampon containing blood.

### EXAMPLE 3

### Growth of S. aureus and Production of TSST-1 by S. aureus in Human Blood

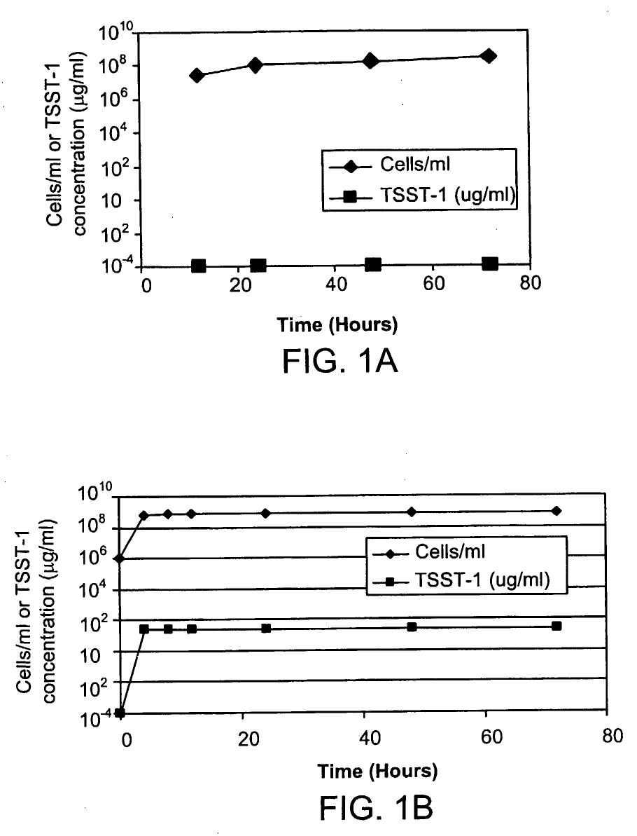

S. aureus MN8 bacteria were added to whole human blood or control beef heart (BH) medium and the mixtures were incubated for 3 days with aeration (shaking at 200 RPM (revolutions per minute)) at 37° C. S. aureus growth and TSST-1 production in the blood (FIG. 1A) and BH medium (FIG. 1B) were compared at various time points. No TSST-1 was detectable in the blood over the whole 72-hour test period. In contrast, after only 4 hours in BH medium, the maximum level of TSST-1 production (24 μg/ml) was reached. The S. aureus grew in both the blood and the BH medium from about 10⁷ cells/ml to about 2×10⁹/ml. These studies indicate that human blood contains one or more mediators that prevent TSST-1 production. Similar results were obtained from human blood that was shown affirmatively to lack antibodies specific for TSST-1. This observation indicated that the failure to detect TSST-1 production in blood was not due to TSST-1-specific antibodies absorbing out synthesized TSST-1 or preventing its synthesis by the bacteria.

Studies performed in essentially the same way as those described in the previous paragraph indicated that the production of staphylococcal enterotoxins B and C (SEB and SEC) by S. aureus strains MNDON and MNNJ were inhibited by human blood.

### EXAMPLE 4

### Purification and Characterization of Exotoxin Inhibitory Factor (EIF)

Experiments were initiated to purify and characterize the above-described EIF.

(a) A menses-saturated tampon from a healthy volunteer and 20 ml of distilled water were added to a 60 ml syringe and the substantially all the fluid contents of the syringe were squeezed out of the syringe using the syringe plunger into a centrifuge tube. After removal of particulate material from the eluate (by centrifugation at 1000×g for 30 minutes), the eluate was tested for microbial contamination by plate counting. No aerobic microbes were detected. The eluate was then subjected to sequential ethanol precipitation. Four fractions containing material precipitable by 25% ethanol (0%-25% ethanol precipitable material), 50% ethanol (25%-50% ethanol precipitable material), and 75% ethanol (50%-75% precipitable material). Precipitated material was pelleted by centrifugation and lyophilized. The resulting precipitates were dissolved in 4 ml of distilled water. Aliquots of each sample and nonprecipitable material were tested for inhibition of TSST-1 production by the MN8 strain of S. aureus (10⁷/ml) after addition to an equal volume of ½ diluted Todd Hewitt medium and culturing for 8 hours at 37° C., with shaking. TSST-1 inhibitory activity was detected primarily in the 0-25 and 25-50% ethanol precipitates and not in the 50-75% and nonprecipitable fractions. In that larger molecules are in general precipitated by lower amounts of ethanol, it is likely that the EIF includes at least one molecule of relatively high molecular weight.

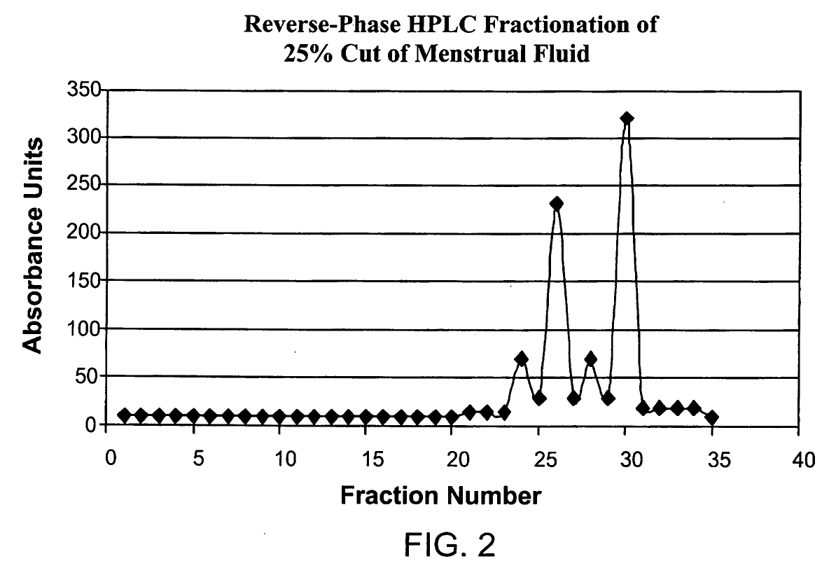

The remainder of the 0-25% ethanol precipitated sample was lyophilized and subjected to reverse phase HPLC with a gradient from 0 to 85% acetonitrile (FIG. 2). One ml fractions were collected, lyophilized, reconstituted to 1 ml with distilled water, and tested for inhibitory activity as described above. Fraction 31 was pink and the only fraction having activity.

(b) Heparinized (50 U/ml) human blood (120 ml) was mixed with 480 ml pyrogen free water to lyse red cells. After a 30-minute incubation at room temperature, 600 ml (50% final volume) of absolute ethanol were added and the mixture which was stirred overnight at 4° C. The precipitate was pelleted by centrifugation (10,000×g, 30 min). The ethanol was evaporated from the surface of the precipitate by placing the centrifuge bottles flat under a laminar flow hood for 30 minutes. The precipitate was then dissolved in 120 ml of pyrogen free water. Insoluble material was removed by centrifugation (10,000×g for 30 minutes). One half of the resulting solution (60 ml) was subjected to a preparative thin layer isoelectric focusing (IEF) procedure. The sample was applied to two identical IEF gels containing pH gradients of pH 3 to pH 10 and the IEF was run overnight.

Testing of the fluid prior to electrofocusing against the mTSS S. aureus isolate MN8 showed that it was inhibitory to TSST-1 production (Table 1). The indicated volumes of the fluid were mixed with 0.25 ml of Todd Hewitt broth containing approximately 1×10⁶/ml S. aureus bacteria and phosphate buffered saline (PBS) to a final volume of 1 ml. The mixtures were incubated for 6 hours and then tested for levels of TSST-1 and numbers of S. aureus. At the highest concentration tested, the fluid was also slightly inhibitory of S. aureus growth.

TABLE 1
Ability of lysed human blood (unfractionated) to inhibit
mTSS S. aureus growth and TSST-1 production.

| Amount tested | | CFU/ml | TSST-1 (ug/ml) |
|---|---|---|---|
| 0.5 | ml | 7.1 × 10⁸ | None Detected |
| 0.1 | ml | 2.1 × 10⁹ | None Detected |
| 0.01 | ml | 2.1 × 10⁹ | None Detected |
| 0 | ml | 2.1 × 10⁹ | 10 |

CPU = colony forming units; None Detected = <0.01 μg; all assays were performed in 1 ml final volume of ¼ diluted Todd Hewitt broth (final concentration) balanced with phosphate buffered saline and lysed human blood.

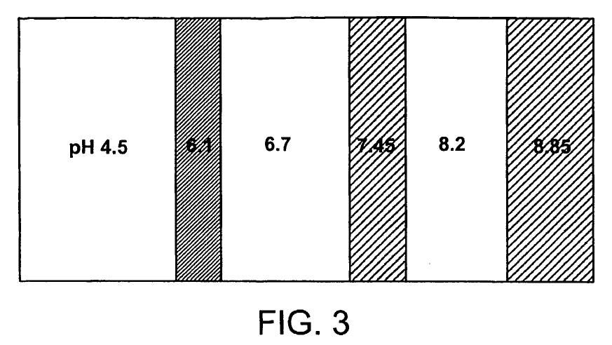

Segments corresponding to colored bands and non-colored regions of the gel between and/or next to colored bands were cut out of the gels. FIG. 3 is a diagram of one of the gels and indicates the positions of the colored bands and non-colored regions between and/or next to colored bands that were cut from the gel. A total of six gel segments were cut from each of the two gels and corresponding gel segments from the two gels were pooled prior to further processing. The contents of each segment was eluted from the gel matrix by filtration through glass wool with pyrogen free water. Elution was performed such that 40 ml of eluate were collected from each sample. The pH of each eluate was determined and each eluate was dialyzed in 12,000 to 14,000 molecular weight cut-off dialysis tubing against 2 liters of water for two days to remove ampholytes. The samples were found to have pH's of 4.5, 6.1, 6.7, 7.45, 8.2 and 8.85 (FIG. 3). The pH 6.1 sample corresponded to a gel band with a brown-green color, the pH 7.45 sample corresponded to a gel band with an intense red color (indicating that hemoglobin was in this gel band), and the pH 8.85 band had a blue-gray color. Aliquots (0.5 ml and 0.1 ml) of each eluate was tested for ability to inhibit TSST-1 production by S. aureus MN8 bacteria and growth of S. aureus MN8 bacteria by a method essentially the same as that described above for testing of the unfractionated fluid. The only sample showing substantial inhibition of TSST-1 was the pH 7.45 sample (Table 2). Inhibition of TSST-1 production was detected when both 0.1 ml and 0.5 ml of the eluate were tested.

These findings indicate the above-described blood EIF includes at least one protein with a pI of 7-8.

TABLE 2
Ability of IEF fractions of human blood to inhibit growth
of mTSS isolate MN8 and prevent TSST-1 synthesis.

| Sample tested (pH) | CFU/ml | TSST-1 (ug/ml) |
|---|---|---|
| 4.5 | 1.5 × 10⁹ | 10 |
| 6.1 | 1.3 × 10⁹ | 10 |
| 6.7 | 1.0 × 10⁹ | 10 |
| 7.45 (0.1 ml) | 7.7 × 10⁸ | 5 |
| 7.45 | 2.9 × 10⁸ | None Detected |
| 8.2 | 1.5 × 10⁹ | 10 |
| 8.85 | 1.6 × 10⁹ | 10 |
| Control media alone | 1.5 × 10⁹ | 10 |

Note:
CPU = colony forming units, None Detected = <0.01 μg; all assays were performed in 1 ml final volume of ¼ diluted Todd Hewitt broth (final concentration) balanced with phosphate buffered saline and IEF sample. 0.5 ml of all IEF samples were added to the 1 ml (final volume) cultures. 0.1 ml of the pH 7.45 sample was also tested.

A number of embodiments of the invention have been described. Nevertheless, it will be understood that various modifications may be made without departing from the spirit and scope of the invention. Accordingly, other embodiments are within the scope of the following claims.

1. An isolated factor that is identical to a factor in blood that inhibits the production of an exotoxin by a bacterium.

2. The factor of claim 1, wherein the factor in blood comprises a protein with an isoelectric point (pI) of 7-8.

3. The factor of claim 1, wherein the bacterium is a Staphylococcus.

4. The factor of claim 3, wherein the Staphylococcus is S. aureus.

5. The factor of claim 1, wherein the exotoxin is toxic shock syndrome toxin 1 (TSST-1).

6. The composition of claim 1, wherein the exotoxin is α-hemolysin or a staphylococcal enterotoxin.

7. A composition comprising:
a medical or hygienic device; and
an isolated factor that is identical to a factor in blood that inhibits the production of an exotoxin by a bacterium.

8. The composition of claim 7, wherein all or part of the surface of the device is coated with or impregnated with the isolated factor.

9. The composition of claim 7, wherein the device is a vaginal tampon.

10. The composition of claim 7, wherein the device is a surgical suture, a surgical bandage, a surgical dressing, or an osmotic pump.

11. The composition of claim 7, wherein the device is an ostomy device.

12. The composition of claim 7, wherein the device comprises a matrix for tissue repair.

13. A composition comprising:
a pharmaceutical carrier or excipient; and
an isolated factor that is identical to a factor in blood that inhibits the production of an exotoxin by a bacterium

14. A method of treating or preventing the symptoms of a bacterial infection, the method comprising delivery to a tissue or organ of a vertebrate subject of an isolated factor that is identical to a factor in blood that inhibits the production of a bacterial exotoxin, wherein the tissue or organ is, or is in danger of being, infected with a bacterium.

15. A method of preventing the symptoms of a bacterial infection, the method comprising:
providing a medical or hygienic device;
providing an isolated factor that is identical to a factor in blood that inhibits the production of a bacterial exotoxin;
applying the device to a vertebrate subject; and
administering to the vertebrate subject the isolated factor.

16. The method of claim 15, wherein the device is coated or impregnated with the isolated factor and the applying of the device and the administration of the factor occurs simultaneously.

17. The method of claim 15, wherein the device is a vaginal tampon.

18. An article of manufacture comprising packaging material and, within the packaging material, a composition comprising an isolated factor that is identical to a factor in blood that inhibits the production of a bacterial exotoxin, wherein the packaging material comprises instructions that indicate that the composition can be used for preventing or inhibiting the symptoms of a bacterial infection.
